(19) United States
(12) Patent Application Publication
(10) Pub. No.: US 2017/0143664 A1
(43) Pub. Date: May 25, 2017

(54) CANNABINOID FORMULATION FOR THE SEDATION AND/OR ANAESTHETIZING OF A HUMAN OR ANIMAL

(71) Applicant: Charles E. Ankner, West Palm Beach, FL (US)

(72) Inventor: Charles E. Ankner, West Palm Beach, FL (US)

(21) Appl. No.: 15/412,211

(22) Filed: Jan. 23, 2017

Related U.S. Application Data
(63) Continuation-in-part of application No. 14/820,507, filed on Aug. 6, 2015, now Pat. No. 9,585,867.
Application No. 14/820,507 is a continuation-in-part of application No. 15/243,439, filed on Aug. 22, 2016.
Application No. 15/243,439 is a continuation-in-part of application No. 14/820,507, filed on Aug. 6, 2015, now Pat. No. 9,585,867.

(51) Int. Cl. A61K 31/352; A61K 31/05; A61K 45/06; A61K 9/00

(52) U.S. Cl. CPC A61K 31/352; A61K 9/0019; A61K 9/007; A61K 9/0053; A61K 31/05; A61K 45/06

(57) ABSTRACT

A method of sedating and/or anesthetizing a human or animal recipient by administering a formulation including a cannabinoid to the recipient thereby rendering the recipient sedated and/or dissociatively unconscious without irreparable harm to or the death of the recipient.

### CROSS REFERENCE TO RELATED APPLICATION

The present application is a continuation-in-part of allowed U.S. patent application Ser. No. 14/820,507, filed Aug. 6, 2015, entitled CANNABINIOD FORMULATION FOR THE SEDATION OF A HUMAN OR ANIMAL, published as U.S. Patent Application Publication No. 2015/0342922 A1, and U.S. patent application Ser. No. 15/243,439 filed Aug. 22, 2016, entitled FORMULATION DELIVERY SYSTEM. Both applications are herein incorporated by reference in their entirety.

### FIELD OF THE INVENTION

The present inventive method relates to a pharmaceutical sedation formulation for quickly and safely sedating and/or anesthetizing a human or an animal. The formulation may include at least one cannabinoid from or derived from plants of the Cannabaceae sensu stricto family, and more specifically plants of the C. Cannabis L. genera.

### BACKGROUND OF THE INVENTION

“In the beginning God made heaven and earth. . . . Then God said, ‘Behold, I have given you every seed-bearing herb that sows seed on the face of all the earth, and every tree whose fruit yields seed; to you it shall be for food. I also give every green plant as food for all the wild animals of the earth, for all the birds of heaven, and for everything that creeps on the earth in which is the breath of life.’ It was so. Then God saw everything He had made, and indeed, it was very good. So evening and morning were the sixth day.” Book of Genesis, Chap 1:1, 29-31, commonly attributed to “the Yahwist”, circa 5^{th} Century B.C.E, as translated and interpreted in The Orthodox Study Bible: Ancient Christianity Speaks to Today's World, Thomas Nelson Publishing, 2008, USA.

“. . . the greatest service which can be rendered to any country is to add a useful plant to its culture; especially a bread grain, next in value to bread, is oil.”, Thomas Jefferson, 3^{rd} President of the United States of America, Memorandum of Services to My Country, 1800, Charlottesville, Va. USA.

“Damn it Charles, no damn good will ever come of this cannabis crap! Plus, it's illegal!” Excited utterance of Frank G. Ankner, father of instant inventor, 1978, Lake Worth, Fla. USA.

Since antiquity, the Cannabaceae sensu stricto (“s.s.”) family of plants have had a wide variety of innovative uses, with some varieties being used for and as food, spice, and ceremonial purposes as early as 8000 B.C.E. Modern uses of the Cannabaceae s.s. family include; varieties being cultivated for plant fiber used in almost innumerable products, varieties being cultivated containing flavonoid and aromatic substances used in the production of beer and in fragrances, varieties being cultivated for human and animal consumption, varieties being cultivated for oil as illumination and lubrication, and being cultivated for oil as bio-fuel replacements for fossil-fuel, and varieties cultivated which contain powerful antimicrobial substances used as sanitizers, antibiotics, and being researched as anti-cancer agents.

Many cultural anthropologists and ethnobotanists hold that C. cannabis L. varieties are among the first plants cultivated by humanity. Modernly, C. cannabis L. varieties are cultivated and utilized extensively and world-wide. Stems, branches, and leaves are used for plant fiber and as biofuel; sprouts and seeds as food-stocks; seeds for inexpensive lubrication and illumination oil, and also as biofuel; flowers for aromatic, recreational, ritual, sacramental, and medicinal purposes; and flowers and roots for medicinal and pharmaceutical formulations.

Recently, substances in some C. cannabis L. varieties have been used to effectively eradicate both MRSA and ORSA bacterium (Methicillin-Resistant Staphylococcus aureus and Oxacillin-Resistant Staphylococcus aureus), occurring both in and ex vivo. MRSA and ORSA are both extremely virulent, antibiotic resistant strains of bacterium which sicken millions and cause hundreds of thousands of deaths per-year world-wide; particularly in industrialized nations. Research continues into using C. cannabis L. variety substances as and in sanitizers and antibiotics which kill pathogens like MRSA and ORSA, and other drug resistant pathogens.

Although never developed into effective weapons systems, psychochemical warfare theory and research, along with overlapping mind control drug research, was secretly pursued in the mid-20th century by the U.S. Military and Central Intelligence Agency, in the context of the Cold War. These research programs were ended when they came to light and generated controversy in the 1970s. The degree to which the Soviet Union developed or deployed similar agents during the same period remains largely unknown. This course of human events during that time hindered or prohibited cannabis, cannabis-derived, or synthetic-cannabinoids from being developed into safe and effective non-lethal sedatives and non-lethal psychochemical weapons. In the 1970s, with the U.S. categorizing cannabis as a Schedule 1 Controlled Substance, touting cannabis as an effective and safe sedation or psychochemical warfare agent would have been prohibited by then public policy and law. Possibly fifty years later, executive governmental agencies, legislatures, law enforcement, civilian defenders, and medical science may now be amenable to just such an effective and safe method of sedating and/or anesthetizing a human or animal.

In the fields of veterinary science, zoology, zoo keeping, animal control, and in many related fields of endeavor, so-called “tranquilizing” apparatus, formulations, and methods are well known and widely used.

It is contemplated that the fields of medicine, veterinary medicine and science, military combat, law enforcement, corrections, emergency response, mass casualty response, and similar fields of endeavor may benefit from cannabinoid sedative formulations, or a cannabinoid being added to or administered with known sedative formulations for medical, scientific, and industrial purposes. Other cannabinoid sedative formulations may be also used for scientific and industrial use improvement and purposes.

Cannabinoids were first discovered in the 1940s when cannabidol (herein “CBD”) and cannabinol (herein “CBN”) were identified and designated. The structure of tetrahydrocannabinol (herein “THC”) was not identified and designated until 1964.

Due to molecular similarity and ease of synthetic conversion, CBD was originally believed to be a natural precursor to and of THC. However, it is now known that CBD and THC are produced independently in the cannabis plant from the precursor cannabigerol (“CBG”). At present, at least 85 different cannabinoids have been isolated and identified from cannabis plants.

Cannabinoids are a class of diverse chemical compounds that among other actions, act on cannabinoid receptors in cells that repress neurotransmitter release in the brains of humans and animals. Ligands have at least one donor atom with an electron pair used to form covalent bonds with the central atom. Ligands for these receptor proteins include endo-cannabinoids (produced naturally in the body), phyto-cannabinoids (found in cannabis and some other plants), and synthetic-cannabinoids (those manufactured artificially).

The most notable cannabinoid is the phyto-cannabinoid THC which is thought to be the primary psychoactive component of cannabis. CBD and CBN are other major cannabinoids of C. Cannabis L plants. It is believed there are yet unknown phyto-cannabinoids to be isolated from cannabis which may exhibit varied effects and affects on and in humans and animals.

Cannabis, and other phyto-cannabinoid producing plants, exhibit wide variation in the quantity, quality, and type of cannabinoids they produce. The mixture of phyto-cannabinoids produced by a plant is typically known as the plant's phyto-cannabinoid “profile” or “presentation”. Selective breeding has been used to influence plant genetics and modify the phyto-cannabinoid presentation. For example, strains that are used as fiber (commonly called industrial hemp) are bred such that they are low in psychoactive chemicals like THC. Strains used in medicine are often bred for high CBD content, and strains used for recreational purposes are usually bred for high THC content, or for a specific desired phyto-cannabinoid balance or profile.

Quantitative analysis of a plant's phyto-cannabinoid profile is often determined by gas chromatography, or more reliably gas chromatography combined with mass spectrometry. Liquid chromatography techniques are also possible, and unlike gas chromatography methods can differentiate between the acid and neutral forms of a phyto-cannabinoid. There have been attempts to systematically monitor the phyto-cannabinoid profile of cannabis over time, but their accuracy has been impeded by prohibitive controlled substance classification status of the cannabis plant in many countries.

Before the 1980s, it was speculated that phyto-cannabinoids produced their physiological and psychoactive effects via nonspecific interaction with cell membranes, instead of in reality interacting with specific cell membrane bound receptors. Discovery of the first cannabinoid receptors in the 1980s resolved this debate. Cannabinoid receptors are common in animals, and have been found in mammals, birds, fish, and reptiles. At present, there are two known types of cannabinoid receptors designated CB1 and CB2—with scientific evidence mounting of more cannabinoid receptors yet to be identified. CB1 receptors are found primarily in the brain, and more specifically in the basal ganglia and limbic system including the hippocampus. CB1 receptors are also found in the cerebellum. The human brain has more cannabinoid receptors, both CB1 and CB2, than any other G protein-coupled receptor (“GPCR”) type. Both human male and female reproductive systems also include CB1 receptors.

CB2 receptors are predominantly found in the immune system, and in immune-derived cells of humans and animals—with the greatest density being in the spleen. While found only in the peripheral nervous system, some studies indicate that CB2 is expressed by a subpopulation of microglia in the human cerebellum. CB2 receptors appear to be responsible for the known anti-inflammatory and possibly other therapeutic effects and affects of cannabis.

Cannabis-derived phyto-cannabinoids are primarily concentrated in viscous resin produced in structures known as “glandular trichomes” of the cannabis plant. All phyto-cannabinoid classes are thought to be derived from CBG type compounds and differ mainly in the way this precursor is cyclized. Classical phyto-cannabinoids are derived from their respective 2-carboxylic acids (2-COOH) by decarboxylation (catalyzed by heat, light, or alkaline conditions). These include but are not limited to CBG (Cannabigerol), CBC (Cannabichromene), CBL (Cannabicyclol), CBV (Cannabivarin), THCV (Tetrahydrocannabivarin), CBDV (Cannabidivarin), CBCV (Cannabichromevarin), CBGV (Cannabigerovarin), and CBGM (Cannabigerol monomethyl ether).

THC is the primary psychoactive component of the cannabis plant. Delta-9-tetrahydrocannabinol and delta-8-tetrahydrocannabinol mimic the action of anandamide (“AEA”), a neurotransmitter produced naturally in the body of humans and animals. These two phyto-cannabinoids produce the classic psychoactive affects and effects associated with cannabis by binding to CB1 receptors in the brain. THC appears to act as an analgesic to ease moderate-to-severe pain, and act as a neuroprotective while also offering the potential to reduce neuroinflammation and to stimulate neurogenesis. THC seems to have approximately equal affinity for CB1 and CB2 receptors.

CBD is not psychoactive and at first was thought to not affect the psychoactivity of THC. However, recent evidence shows that cannabis users prone to psychosis while using high THC to CBD ratio cannabis had fewer and less extreme psychotic-like symptoms using high CBD to THC ratio cannabis. Some research suggests that the antipsychotic effects of CBD potentially represent a novel mechanism in the treatment of schizophrenia and other affective disorders. CBD has little affinity for CB1 and CB2 receptors, but acts as an indirect antagonist of cannabinoid agonists. Recently, CBD was found to be an antagonist at the putative new cannabinoid receptor GPR55, a GPCR expressed in the caudate nucleus and putamen of the brain. CBD appears to relieve convulsion, inflammation, anxiety, and nausea, and has a greater affinity for the CB2 receptor than for the CB1, although the overall affinity to both is weak. CBD shares a precursor with THC and is the main cannabinoid in low-THC cannabis strains. CBD also apparently plays a role in preventing the short-term memory loss associated with THC in mammals. CBD has also been shown to act as a 5-HT1A receptor agonist. Some beneficial effects observed from 5-HT1A receptor activation are decreased aggression, increased sociability, decreased impulsivity, inhibition of drug-seeking behavior, facilitation of sex drive and arousal, inhibition of penile erection, decreased food intake, prolonged REM sleep latency, and reversal of opioid-induced respiratory depression.

CBN is primarily a product of THC degradation, and there is usually little CBN in living or freshly harvested cannabis. CBN content increases as THC degrades in storage and with exposure to light and air. CBN is only mildly psychoactive, and its affinity to the CB2 receptor is higher than to the CB1.

Many ethnobotanists, organic chemists, biochemists, and medical professionals consider THC, CBD, and CBN to be the “big three” of phyto-cannabinoids and cannabis, and of which's ratio primarily effects the profile or presentation of a specific cannabis variety. The overall effects and affects of a particular cannabis strain caused by the interplay and ratio of THC, CBD, and CBN is generally and commonly referred to as the strain's “entourage effect”.

Cannabigerol (“CBG”) is non-psychotomimetic but still impacts the overall effects and affects of cannabis. CBG acts as a α2-adrenergic receptor agonist, 5-HT1A receptor antagonist, CB1 receptor antagonist, and also binds to the CB2 receptor.

Tetrahydrocannabivarin (“THCV”) is prevalent in certain central Asian and southern African strains of cannabis. It is an antagonist of THC at CB1 receptors and attenuates the psychoactive effects of THC. The psychoactive effects of THCV in cannabis and cannabis formulations are not yet well characterized. Unlike THC, CBD, and cannabichromene (“CBC”), THCV doesn't begin as cannabigerolic acid (“CBGA”). Instead of combining with olivetolic acid to create CBGA, geranyl pyrophosphate joins with divarinolic acid, which has two less carbon atoms. The result is cannabigerovarin acid (“CBGVA”). Once CBGVA is created, the process continues as it would for THC. CBGVA is broken down to tetrahydrocannabivarin carboxylic acid (“THCVA”).

Cannabidivarin (“CBDV”) usually comprises a minor part the cannabis phyto-cannabinoid profile. Enhanced levels of CBDV have been reported in feral cannabis plants of the northwest Himalayas and in hashish from Nepal. GW Pharmaceuticals is actively developing a CBDV based formulation due to CBDV's demonstrated neurochemical pathway for previously observed antiepileptic and anticonvulsive affects.

Cannabichromene (“CBC”) is non-psychoactive, does not affect the psychoactivity of THC, and is more common in tropical cannabis varieties. CBC exhibits anti-inflammatory and analgesic properties. Evidence suggests that CBC may play a role in anti-inflammatory and anti-viral effects, and may contribute to the overall analgesic effects of cannabis. One study in 2010 showed that CBC along with CBD and THC has antidepressant affects. Another study showed that CBC helps promote neurogenesis.

Cannabinoid production in cannabis starts when an enzyme causes geranyl pyrophosphate and olivetolic acid to combine and form CBGA. Next, CBGA is independently converted to either CBG, THCA, CBDA or CBCA by four separate synthase, FAD-dependent dehydrogenase enzymes. There is no evidence for enzymatic conversion of CBDA or CBD to THCA or THC. For the propyl homologues (THCVA, CBDVA and CBCVA), there is an analogous pathway that is based on CBGVA from divarinolic acid instead of olivetolic acid.

Each of the cannabinoids above may be in different forms depending on the position of the double bond in the alicyclic carbon ring. There is potential for confusion because there are different numbering systems used to describe the position of this double bond. Under the dibenzopyran numbering system widely used today, the major form of THC is called Δ9-THC, while the minor form is called Δ8-THC. Under an alternate terpene numbering system, these same compounds are labeled Δ1-THC and Δ6-THC, respectively.

Accordingly, and herewithin, tetrahydrocannabinol and/or “THC” shall be defined to include the delta-9-tetrahydrocannabinol, delta-8-tetrahydrocannabinol, delta-1-tetrahydrocannabinol, and the delta-6-tetrahydrocannabinol designations; as well as their metabolites, including 11-OH-delta-9-tetrahydrocannabinol, 11-OH-delta-8-tetrahydrocannabinol, 11-OH-delta-1-tetrahydrocannabinol, and 11-OH-delta-6-tetrahydrocannabinol designations.

Most classical cannabinoids are twenty-one-carbon compounds. However, some do not follow this rule primarily because of variation in the length of the side-chain attached to the aromatic ring. In THC, CBD, and CBN, this side-chain is a pentyl (five-carbon) chain. In the most common homologue, the pentyl chain is replaced with a propyl (three-carbon) chain. Cannabinoids with the propyl side-chain are named using the suffix varin, and are designated, for example, THCV, CBDV, or CBNV.

Phyto-cannabinoids are known to occur in several plant species besides cannabis. These include but are not limited to echinacea purpurea, echinacea angustifolia, echinacea pallida, acmella oleracea, helichrysum umbraculigerum, and radula marginata. The best-known cannabinoids that are not derived from cannabis are the lipophilic alkamides (alkylamides) from the echinacea species, most notably the cis/trans isomers dodeca-2E, 4E, 8Z, 10E/Z-tetraenoic-acid-isobutylamide. At least 25 different alkylamides have been identified, and some have shown affinities to CB2 receptors. In echinacea species, cannabinoids are found throughout the plant structure but are most concentrated in the roots and flowers. Yangonin found in the kava plant is a ligand to the CB1 receptor. Tea (camellia sinensis) catechins also have an affinity for human cannabinoid receptors. A widespread dietary cannabinoid, beta-caryophyllene, a component from the essential oil of cannabis and other medicinal plants, has also been identified as a selective agonist of peripheral CB2 receptors in vivo. Black truffles also contain anandamide.

Most phyto-cannabinoids are nearly insoluble in water, but are soluble in lipids, alcohols, and other non-polar organic solvents.

Cannabinoids can be administered by many methods typically including but not limited to smoking, vaporizing, ingestion, transdermal sorption, sublingual sorption, or other mucosa sorption. Once in the body, most cannabinoids are metabolized in the liver, especially by cytochrome P450 mixed-function oxidases, mainly CYP 2C9. Thus, supplementing the inventive formulation with CYP 2C9 inhibitors may lead to extended or enhanced intoxication, incapacitation, or immobilization.

Cannabinoids can be separated from cannabis or other plants by extraction with organic solvents. Hydrocarbons and alcohols are often used as solvents. However, these solvents are extremely flammable and many are toxic. Butane may also be used, which evaporates extremely quickly. Supercritical solvent extraction with carbon dioxide is an alternative technique. Although this process requires high pressures, there is minimal risk of fire or toxicity, solvent removal is simple and efficient, and extract quality can be well controlled. Once extracted, cannabinoid blends can be separated into individual components using wiped film vacuum distillation or other distillation techniques. However, to produce high-purity cannabinoids, chemical synthesis or semi-synthesis is generally required.

Endo-cannabinoids are substances produced from within the body that activate cannabinoid receptors. After discovery of the first cannabinoid receptor in 1988, scientists began searching for an endogenous ligand for the receptor.

Endo-cannabinoids serve as intercellular “lipid messengers”, signaling molecules that are released from one cell and activating the cannabinoid receptors present on other nearby cells. Although in this intercellular signaling role they are similar to the well-known monoamine neurotransmitters, such as acetylcholine and dopamine, endo-cannabinoids differ in numerous ways. For example, endo-cannabinoids are used in retrograde signaling between neurons. Furthermore, endo-cannabinoids are lipophilic molecules that are not very soluble in water. They are not stored in vesicles, and exist as integral constituents of the membrane bilayers that make up cells. Endo-cannabinoids are believed to be synthesized “on-demand” rather than made and stored for later use. The mechanisms and enzymes underlying the biosynthesis of endo-cannabinoids remain elusive and continue to be an area of active research.

Conventional neurotransmitters are released from a “presynaptic” cell and activate appropriate receptors on a “postsynaptic” cell, where presynaptic and postsynaptic designate the sending and receiving sides of a synapse, respectively. Endo-cannabinoids, on the other hand, are described as retrograde transmitters because they most commonly travel “backward” against the usual synaptic transmitter flow. They are, in effect, released from the postsynaptic cell and act on the presynaptic cell, where the target receptors are densely concentrated on axonal terminals in the zones from which conventional neurotransmitters are released. Activation of cannabinoid receptors temporarily reduces the amount of conventional neurotransmitter released. This endo-cannabinoid mediated system permits the postsynaptic cell to control its own incoming synaptic traffic. The ultimate effect on the endo-cannabinoid-releasing cell depends on the nature of the conventional transmitter being controlled. For instance, when the release of the inhibitory transmitter GABA is reduced, the net effect is an increase in the excitability of the endo-cannabinoid-releasing cell. On the converse, when release of the excitatory neurotransmitter glutamate is reduced, the net effect is a decrease in the excitability of the endo-cannabinoid releasing cell.

Endo-cannabinoids are hydrophobic molecules, they cannot travel unaided for long distances in the aqueous medium surrounding the cells from which they are released, and therefore act locally on nearby target cells. Hence, although emanating diffusely from their source cells, they have much more restricted spheres of influence than do hormones which can affect cells throughout the body.

In 1992 the first such endo-cannabinoid compound was identified as arachidonoylethanolamine, and named anandamide (“AEA”). AEA is derived from arachidonic acid and has a pharmacology similar to THC, although its chemical structure is different. AEA has an affinity for CB1 receptors and to a lesser extent CB2, where it acts as a partial agonist. AEA is about as potent as THC at the CB1 receptor, and is found in nearly all tissues in a wide range of animals. AEA has also been found in plants, including small amounts in cocoa beans from which chocolate is made. Two analogs of AEA, 7, 10, 13, and 16-docosatetraenoylethanolamide and homo-γ-linolenoylethanolamine, have similar pharmacology. All of these are members of a family of signaling lipids called N-acylethanolamines, which also includes the noncannabimimetic palmitoylethanolamide and oleoylethanolamide, which possess anti-inflammatory and orexigenic effects, respectively. Many N-acylethanolamines have also been identified in certain other plant seeds and also in mollusks.

Another endo-cannabinoid, 2-arachidonoylglycerol (“2-AG”), binds to both the CB1 and CB2 receptors with similar affinity, acting as a full agonist at both. 2-AG is present at significantly higher concentrations in the brain than AEA, causing some controversy whether 2-AG rather than AEA is chiefly responsible for endo-cannabinoid signaling in vivo. In particular, one in vitro study suggests that 2-AG is capable of stimulating higher G-protein activation than AEA, although the physiological implications of this finding are not yet known.

Discovered in 2000, N-arachidonoyl dopamine (“NADA”) preferentially binds to the CB1 receptor. Like AEA, NADA is also an agonist for the vanilloid receptor subtype 1 (TRPV1), a member of the vanilloid receptor family. Outside the food industry, vanilloids which act at TRPV1 are used in so-called “pepper-spray” and/or other mace formulations.

In 2001, a fourth, ether type endo-cannabinoid, 2-arachidonyl glyceryl ether (“noladin ether”) was isolated from porcine brain. Prior to this discovery, noladin ether had been synthesized as a stable analog of 2-AG; indeed, some controversy remains over 2-AGs classification as an endo-cannabinoid, as another group failed to detect the substance at “any appreciable amount” in the brains of several different mammalian species. Noladin ether binds to the CB1 receptor and causes sedation, hypothermia, intestinal immobility, mild reduced sensitivity to pain in mice, and binds weakly to the CB2.

A fifth endo-cannabinoid, virodhamine, or O-arachidonoyl-ethanolamine (“OAE”), was discovered in 2002. Although it is a full agonist at CB2 and a partial agonist at CB1, it behaves as a CB1 antagonist in vivo. In rats, OAE was found to be present at comparable or slightly lower concentrations than AEA in the brain, but peripherally in two-to-nine fold higher concentrations.

Recent evidence has highlighted lysophosphatidylinositol (“LPI”) as the endogenous ligand to novel endo-cannabinoid receptor GPR55, making it a strong contender as the sixth endo-cannabinoid.

Historically, laboratory synthesis of cannabinoids were often based on the structure of herbal or phyto-cannabinoids, and a large number of analogs have been produced and tested. Synthetic-cannabinoids are particularly useful in experiments to determine the relationship between the structure and activity of cannabinoid compounds, by making systematic and incremental modifications of cannabinoid molecules. When synthetic-cannabinoids are used recreationally, they present significant health dangers to users. In the period of 2012 through 2014, over 10,000 contacts to poison control centers in the United States were related to use or abuse of synthetic-cannabinoids.

Medications containing natural or synthetic-cannabinoids or cannabinoid analogs include: Dronabinol (Marinol), which is Δ9-THC used as an appetite stimulant, anti-emetic, and analgesic; Nabilone (Cesamet, Canemes), a synthetic cannabinoid and an analog of Marinol; Rimonabant (SR141716), a selective CB1 receptor inverse agonist once used as an anti-obesity drug under the proprietary name Acomplia, and was also used for smoking cessation; CP-55940, produced in 1974 as a synthetic cannabinoid receptor agonist many times more potent than the phyto-cannabinoid THC; Dimethylheptylpyran (DMHP), an analog of phyto-cannabinoid THC; HU-210, about 100 times as potent as phyto-cannabinoid THC; HU-331, a potential anti-cancer drug derived from CBD that specifically inhibits topoisomerase II; SR144528, a CB2 receptor antagonist; WIN 55,212-2, a potent cannabinoid receptor agonist; JWH-133, a potent selective CB2 receptor agonist; Levonantradol (Nantrodolum), an anti-emetic and analgesic, but not currently in use in medicine; and AM-2201, a potent cannabinoid receptor agonist.

Therefore, what is highly desired is a method of and formulation for sedating and/or anesthetizing a human or animal including a cannabinoid; wherein the formulation once administered renders the recipient sedated and/or fully dissociatively unconscious without irreparable harm to or the death of the recipient.

### SUMMARY OF THE INVENTION

Advantageously, CB1 receptors are absent in the mammalian medulla oblongata, the part of the brain stem responsible for autonomic respiratory and cardiovascular function. This is highly advantageous when cannabinoids are used for and as sedative and anesthetic formulations. Affecting or depressing autonomic respiratory and/or cardiovascular function has long been a limiting disadvantage of known sedative and anesthetic formulations. Fortunately, unless introduced at extremely toxic levels, CB1 agonist cannabinoids primarily leave autonomic respiratory and cardiovascular functions in humans and animals unaffected, due to the lack of CB1 receptors in the brain stem medulla oblongata.

It is therefore an object of the present invention to provide a general anesthetic formulation for sedating a human or animal including a cannabinoid, wherein when administered results in a THC blood level in the recipient of approximately twenty-five to fifteen-hundred milligrams per kilogram of body weight (25 mg-1500 mg/kg) and below a dosage which causes irreparable harm to or the death of the recipient; and wherein the recipient once dosed is rendered unconscious for safely and painlessly undergoing surgical and other medical procedures.

It is a further object of the present invention to provide a method of anesthetizing a human or animal including providing a formulation including a cannabinoid, and providing a delivery system capable of dosing a recipient with the formulation which renders the recipient sedated and unconscious without irreparable harm to or the death of the recipient.

Another object of the present invention is to provide a system for anesthetizing a human or animal, the system including a formulation including a cannabinoid which renders a recipient sedated after dosing; and wherein upon dosage of the recipient, a THC blood level is induced of approximately one-quarter-to-one-hundred milligrams per milliliter of whole blood (0.25 mg-100 mg/ml) and below a dosage which causes irreparable harm to or the death of the recipient, and wherein the recipient once dosed may safely and painlessly undergo surgical and other medical procedure.

Another object of the present invention method is to administer the formulation hypodermically, orally, or via inhalation.

Another object of the present invention method is to administer the formulation via inhalation and including at least one anesthetic gas from the group consisting of benzodiapines, diprivan, thiopental, ketamine, desflurane, isoflurane, nitrous oxide, sevoflurane, xenon, and combinations thereof.

Another object of the present invention method is to administer the formulation hypodermically, including at least one dissociative anesthetic from the group consisting of benzodiazepines, barbiturates, opiates, diprivan, and combinations thereof.

Another object of the present invention method is to administer the formulation hypodermically, including at least one antipsychotic from the group consisting of antipsychotic or neroleptic formulations including butyrophenones, phenothiazines, thioxanthenes, atypical antipsychotics, second-generation antipsychotics, and combinations thereof.

Another object of the present invention method is to administer the formulation via a gas, fluid, liquid, semi-solid or a solid.

It is a further object of the present invention method of anesthetizing a human or animal includes providing a formulation having equal amounts of THC, CBD, and CBN.

It is a further object of the present invention method of anesthetizing a human or animal including providing a formulation wherein the amount of THC is greater than that of either the CBD or CBN.

It is a further object of the present invention method of anesthetizing a human or animal including providing a formulation wherein the amount of CBD is greater than that of either the THC or CBN.

It is a further object of the present invention method of anesthetizing a human or animal including providing a formulation wherein the amount of CBN is greater than that of either the THC or CBD.

It is a further object of the present invention method of anesthetizing a human or animal providing a formulation including a CYP 2C9 inhibitor.

It is a further object of the present invention method of anesthetizing a human or animal wherein the delivery system provides the anesthetizing dose of the formulation to the recipient at selected potencies, at selected intervals, and for selected durations.

It is a further object of the present invention method of anesthetizing a human or animal wherein the formulation includes a cannabinoid emulsified in sesame oil, polysorbate 80 or a saline vehicle.

It is a further object of the present invention method of anesthetizing a human or animal wherein the concentration of the cannabinoid in the formation ranges from 15 mg/m I to 40 mg/ml.

It is a further object of the present invention method of anesthetizing a human or animal wherein the amount of the cannabinoid provided to the human or animal ranges from 0.25 mg to 10 mg.

It is a further object of the present invention method of anesthetizing a human or animal wherein the amount of the cannabinoid provided to the human or animal ranges from 10 mg to 100 mg.

It is a further object of the present invention method of anesthetizing a human or animal wherein the cannabinoid formulation includes less than 9200 mg of THC per kg of body weight of the recipient, such that the dose administered to the recipient is a non-lethal dose.

It is a further object of the present invention to provide a method and system of anesthetizing a human or animal, the system including a formulation including a cannabinoid which renders a recipient sedated after dosing; and wherein upon dosage of the recipient, a THC blood level is induced of approximately one-quarter-to-one-hundred milligrams per milliliter of whole blood (0.25 mg-100 mg/ml) and below a dosage which causes irreparable harm to or the death of the recipient, and wherein the recipient once dosed may safely and painlessly undergo surgical and other medical procedures.

### DETAILED DESCRIPTION OF THE INVENTION

The inventive sedation formulation may be primarily a cannabinoid based formulation. However, cannabinoids may be added to known sedative formulations to improve their safety and/or performance. Many and varied cannabinoid formulations may be innovated.

In an exemplary embodiment of the inventive formulation, a sedating dosage of THC may be added to known effective dosages of propofol (diprivan). For healthy adults 55 years or younger, a general intravenous anesthetic infusion of diprivan is 40 mg every 10 seconds until induction onset. For general anesthetic use, a typical dose of diprivan is 2.0-2.5 mg per kilogram of recipient body weight, with a maximum dosage of 250 mg.

By adding an appropriate dose of THC to result in approximately twenty-five to fifteen-hundred milligrams per kilogram of body weight (25 mg-1500 mg/kg) and below a dosage which causes irreparable harm to or the death of the recipient; a recipient once dosed may be rendered unconscious for safely and painlessly undergoing surgical and other medical procedures.

Once dosed, the recipient will be almost instantaneously incapacitated by the diprivan, within seconds, while the THC dose will still further sedate the recipient for approximately another four-to-six hours, without depression of the body's autonomic functions.

If it is unnecessary to “immediately”, or to “near-immediately”, sedate a recipient, the inventive formulation may strictly consist of cannabinoids such as THC.

The lethality of intravenous dosing of THC in humans is typically unknown. As detailed in Marihuana, A Signal of Misunderstanding, a report delivered to the United States Congress by Raymond P. Shafer on Mar. 22, 1972 (herein incorporated by reference in its entirety), in laboratory animals, a dosage that caused death in 50% of subjects (“LD50”) was in units of mg of THC per kg of body weight. In mice and rats, an LD50 THC dose is 28.6 mg per 42.47 kg of body weight. A dosage of approximately 1000 mg of THC per kg of body weight is known to be the lowest intravenous dosage which causes death in laboratory animals. The typical lethal oral dosage of THC is between approximately 225-450 mg per kg of body weight in laboratory animals.

Using intravenous administration, the acute one dose LD50 for THC was 100 mg/kg in dogs and 15.6-62.5 mg/kg in monkeys depending on concentration of the solution. The minimal lethal intravenous dose for dogs, also depending upon concentration, was 25-99 mg/kg, and for monkeys 3.9-15.5 mg/kg.

In contrast to the delayed death observed in rats after oral administration, lethality in rats, dogs, and monkeys after intravenous injection occurred within minutes. When sublethal amounts were injected, central nervous system depression with concomitant behavioral changes similar to those observed after oral doses were observed. However, their onset was more rapid and the intensity of affect more severe with anesthesia, with convulsions noted after injection. Monkeys and dogs that survived the intravenous injection recovered completely within five to nine days.

The only consistent pathological changes noted were in animals which succumb. Pulmonary changes including hemorrhage, edema, emphysema, and generalized congestion were found—and death resulted from respiratory arrest and subsequent cardiac failure. The investigators presumed one mechanism possibly accounting for these findings was due to the concentration of the THC solution and its insolubility in water. Presumably when these highly concentrated solutions mixed with blood, the THC precipitated out of solution. The precipitated foreign material then formed aggregates (or emboli) that were filtered out in the lung capillaries causing a physical blockage of pulmonary blood flow.

Subsequently, intravenous studies were repeated using THC emulsified in a sesame oil, polysorbate 80, or saline vehicles at 15 mg/ml or 40 mg/ml. The emulsions were administered at a uniform rate of 2 ml/15 sec. Doses administered were 1, 4, 16, 64, 92,128, 192 and 256 mg/kg. All monkeys injected with 92 mg/kg or less survived and completely recovered from all effects within two to four days. An analogous intravenous dosage for a 100 kg human would be 9,200 mg (9.2 g) of near-pure THC. All monkeys injected with 128 mg/kg or more succumb within thirty minutes for all but one subject, which took one-hundred-and-eighty minutes to expire. An analogous lethal intravenous dosage for a 100 kg human would be 12,800 mg (12.8 g) of near-pure THC.

Histopathological changes found in the lungs of the deceased monkeys were like those described after the previous intravenous experiment. All monkeys that died exhibited severe respiratory depression and bradycardia within five minutes after injection. Respiratory arrest and subsequent cardiac failure occurred within minutes. Behavioral changes preceding death were salivation, prostration, coma, and tremors.

Behavioral and physiological changes described clinically in the surviving monkeys followed a consistent developmental sequence and were roughly dose related in severity and duration. Onset was fifteen minutes following injection and duration was up to forty-eight hours. Huddled posture and lethargy were the most persistent changes. Constipation, anorexia, and weight loss were noted. Hypothermia, bradycardia, and decreased respiratory rate generally were maximal two-to-six hours post injection. Tremors with motion but not at rest were believed to be caused by peripheral muscle inadequacy.

Enormous intravenous doses of THC, and all THC and concentrated cannabis extracts ingested orally were unable to produce death or organ pathology in large mammals, but did produce fatalities in smaller rodents due to profound central nervous system depression.

The nonlethal oral consumption of 3 g/kg of THC by a dog and monkey would be comparable to a 154-pound human eating approximately forty-six pounds, 21 kg, of one-percent THC cannabis, or ten-pounds of five-percent hashish, at one time. In addition, 92 mg/kg THC intravenously produced no fatalities in monkeys. These doses would be comparable to a 154-pound human smoking at one time almost three pounds (1.28 kg) of one-percent THC cannabis, 250,000 times the usual smoked dose, and over a million times the minimal effective dose assuming fifty-percent destruction of the THC by combustion.

Instant inventor mathematical extrapolation and interpolation indicate the following adult human lethality doses for and of hypodermically injected THC:

100% survival—9200 mg of THC per kg of body weight. 100% lethality—12800 mg of THC per kg of body weight.

Estimated whole blood volume of a 100 kg human male is 7500 mL, or 75 mL of whole blood per kg of body weight.

Converted: 100% survival—122 mg of THC per 1 mL of whole blood. 100% lethality—170 mg THC per 1 mL of whole blood.

During preliminary instant inventor experimentation, dronabinol was administered via bolus intermuscular quadricep injection with a 0.5 mL polysorbate 80 and 10 mL saline carrier:

First Injection—1 mg dronabinol, typical recreational cannabis intoxication symptom onset (euphoria, visual spacial disorientation and slight distortion) within thirty-to-sixty seconds; peaking within five to seven minutes with a ninety-minute duration.

Second Injection—2.5 mg dronabinol, intense and severe recreational cannabis intoxication symptom onset (dissociative euphoria, extreme visual spacial disorientation, audio sensitivity) within ten-to-thirty seconds; peaking within one minute with a three-hour duration.

Third Injection—10 mg dronabinol, severe cannabis intoxication symptom onset (dissociative euphoria, extreme visual spacial disorientation, audio sensitivity) within five seconds, followed by dissociative unconsciousness within thirty-to-sixty seconds lasting ninety-to-one hundred and twenty minutes. Recovery to “first injection state” within two hours.

Forth Injection—25 mg dronabinol, severe cannabis intoxication symptom onset (dissociative euphoria, extreme visual spacial disorientation, audio sensitivity) within two-to-five seconds, followed by dissociative unconsciousness within thirty-to-sixty seconds lasting four hours. Recovery to “first injection state” within six hours.

Forty-eight hours passed between injections, and instant inventor experimentation ended after the fourth injection.

During subsequent instant inventor experimentation, an intermuscular injection of 1000 mg of THC rendered an overall healthy 50 year old, 100 kg male, fully unconscious within 5-15 seconds, with its effects lasting approximately four-to-six hours.

Instant inventor experimentation indicates an assured effective dose of THC for sedation resulting in full dissociative unconsciousness to be approximately 1 to 10 mg of THC per 1 mL of whole blood for an adult human male, and approximately 0.5 to 7.5 mg of THC per 1 mL of whole blood for an adult human female.

Therefore, depending on the emergency and circumstances involved, it is highly desired and an object of the present invention to provide a sedative formulation dose to an adult human weighting between 50 and 120 kg with a formulation including between 250 to 2500 mg of THC to ensure safe, effective, and extremely rapid and full dissociative unconsciousness.

It is also highly desired, contemplated, and a further object of the instant invention to provide a formulation that if an initial or first dosing of between 250 to 2500 mg of THC is insufficient to effectively disassociate a human recipient, an additional or a plurality of doses including between 100 to 250 mg of THC may be safely administered without concern of reaching or exceeding any known or contemplated lethal THC levels.

Formulations of THC may include carriers and or solvents which include sesame oil, polysorbate, polysorbate 80 and/or saline. Other organic and non-toxic solvents and/or surfactants are also contemplated. Additionally, the formulation may contain one or more excipients, such as buffers, alcohols, lipids, ascorbic acid, phospholipids, EDTA, sodium chloride, mannitol, sorbitol, and glycerol, for example.

Other carriers are also contemplated which allow for the THC to be misted to form a solution or a mixture, such that a lipid formulations of the THC may be dosed to effectively sedate a recipient.

While not wishing to be bound by any one theory or combination of theories, the instant inventor has discovered that THC and other cannabinoids cause the quick and/or immediate full dissociative unconsciousness of a human or animal when delivered at high doses, while being safe and non-lethal; and thus may be used for industrial, scientific, and medical purposes.

Thus, evidence from animal studies, human case reports, and instant inventor experimentation indicate that the ratio of an effective-dose to lethal-dose of THC and other cannabinoids is quite large; and much more favorable than that of many other common psychoactive agents including alcohol, barbiturates, and opiates. This effective-to-lethal cannabinoid dosage range may be exploited for medical, scientific, and industrial purposes.

While not wishing to be bound by any one theory or combination of theories, it is believed that, the combination, ratio, delivery system, method, or technique, dosage, dosage timing, dosage sequence, and in combination with other known sedatives; cannabinoids, and specifically THC, CBD, and CBN, may be exploited for industrial, scientific, and medical use.

Relatedly, known antipsychotic compounds may be included in the inventive cannabinoid formulation to prevent or mitigate quick onset and/or violent psychotic reactions to the inventive cannabinoids, especially THC. Such known antipsychotic or neroleptic formulations include but are not limited to butyrophenones, phenothiazines, thioxanthenes, so-called atypical antipsychotics, and so-called second-generation antipsychotics.

Accordingly, this invention is not to be limited by the embodiments as described, since these are given by way of example only and not by way of limitation.

Having thus described several embodiments for practicing the inventive method, its advantages and objectives can be easily understood. Variations from the description above may and can be made by one skilled in the art without departing from the scope of the invention, which is to be determined from and by the following claims.

What is claimed is:

1. A general anesthetic formulation for sedating a human or animal recipient, comprising:
a cannabinoid; and
at least one cannabinoid agent,
wherein when said formulation is administered to the recipient results in a tetrahydrocannabinol blood level in the recipient of greater than twenty-five to fifteen-hundred milligrams per kilogram of body weight (25 mg-1500 mg/kg) and below a dosage which causes irreparable harm to or the death of the recipient; and
wherein the recipient once dosed is rendered unconscious.

2. The formulation of claim 1, wherein the formulation is administered hypodermically.

3. The formulation of claim 1, wherein the formulation is administered via inhalation; and includes at least one anesthetic gas from the group consisting of benzodiapines, diprivan, thiopental, ketamine, desflurane, isoflurane, nitrous oxide, sevoflurane, xenon, and combinations thereof.

4. The formulation of claim 1, wherein the formulation is administered orally.

5. The formulation of claim 1, wherein the formulation includes at least one dissociative anesthetic from the group consisting of benzodiazepines, barbiturates, opiates, diprivan, and combinations thereof.

6. The formulation of claim 1, wherein the formulation includes at least one antipsychotic from the group consisting of antipsychotic or neroleptic formulations including butyrophenones, phenothiazines, thioxanthenes, atypical antipsychotics, second-generation antipsychotics, and combinations thereof.

7. The formulation of claim 1, wherein the formulation comprises a gas, fluid, or liquid.

8. The formulation of claim 1, wherein the formulation comprises a semi-solid or a solid.

9. The formulation of claim 1, wherein the formulation includes tetrahydrocannabinol, cannabidiol, and cannabinol.

10. The formulation of claim 9, wherein the amount of tetrahydrocannabinol is greater than that of either the cannabidiol or cannabinol.

11. The formulation of claim 9, wherein the amount of cannabidiol is greater than that of either the tetrahydrocannabinol or cannabinol.

12. The formulation of claim 9, wherein the amount of cannabinol is greater than that of either the tetrahydrocannabinol or cannabidiol.

13. The formulation of claim 1, wherein the formulation includes a CYP 2C9 inhibitor.

14. The method of claim 1, wherein the delivery system provides the anesthetizing dose of the formulation to the recipient at selected potencies, at selected intervals, and for selected durations.

15. The method of claim 1, wherein the cannabinoid is emulsified in sesame oil, polysorbate 80 or a saline vehicle.

16. The method of claim 1, wherein the concentration of the cannabinoid in the formation ranges from 15 mg/ml to 40 mg/ml.

17. The method of claim 1, wherein the amount of the cannabinoid provided to the human or animal ranges from 0.25 mg to 10 mg.

18. The method of claim 1, wherein the amount of the cannabinoid provided to the human or animal ranges from 10 mg to 100 mg.

19. The formulation of claim 1, wherein the cannabinoid formulation includes less than 9200 mg of tetrahydrocannabinol per kg of body weight of the recipient, such that the dose administered to the recipient is a non-lethal dose.

20. A method of anesthetizing a human or animal recipient comprising:
providing a sedative formulation including a cannabinoid;
providing a delivery system capable of dosing a recipient with the sedative formulation which renders the recipient sedated and unconscious without irreparable harm to or the death of the recipient; and
administering the sedative formulation to the recipient by using the delivery system thereby rendering the recipient unconscious.

21. The method of claim 20, wherein the sedative formulation dose to an adult human weighting between 50 and 120 kg includes between 250 to 2500 mg of tetrahydrocannabinol.

22. The method of claim 21, wherein the initial or first dosing of between 250 to 2500 mg of tetrahydrocannabinol is insufficient to effectively disassociate the recipient, an additional or a plurality of the sedative formulation including between 100 to 250 mg of tetrahydrocannabinol is administered.

23. A system for anesthetizing a human or animal recipient, the system comprising:
a formulation including a cannabinoid which renders the recipient sedated after dosing; and
wherein upon dosage of the recipient, a tetrahydrocannabinol blood level is induced of approximately one-quarter-to-one-hundred milligrams per milliliter of whole blood (0.25-100 mg/ml) and below a dosage which causes irreparable harm to or the death of the recipient, and
wherein the recipient once dosed may safely and painlessly undergo surgical and other medical procedures.
